(19) United States
(12) Patent Application Publication
(10) Pub. No.: US 2024/0123184 A1
(43) Pub. Date: Apr. 18, 2024

(54) CATHETER WITH MULTIPLE WORKING CHANNELS

(71) Applicant: Covidien LP, Mansfield, MA (US)

(72) Inventors: Tina M. Berthiaume, Champlin, MN (US); Nathan J. Knutson, Long Lake, MN (US); John W. Komp, Dillon, CO (US)

(73) Assignee: Covidien LP, Mansfield, MA (US)

(21) Appl. No.: 18/277,472

(22) PCT Filed: Mar. 14, 2022
(86) PCT No.: PCT/US2022/020114

§ 371 Date: Aug. 16, 2023

Related U.S. Application Data
(60) Provisional application No. 63/163,029, filed on Mar. 18, 2021.

(51) Int. Cl. A61M 25/00

(52) U.S. Cl. CPC A61M 25/0032; A61M 2025/0037; A61M 2210/1039

(57) ABSTRACT

A catheter includes a shaft portion defining a non-circular transverse cross-section. The shaft portion includes an exterior sidewall and an interior sidewall defining two or more asymmetrical working channels.

### BACKGROUND

### Technical Field

The present disclosure is generally related to catheters including multiple working channels, and more particularly, catheters including a shaft portion defining a non-circular transverse cross-section, the shaft portion including multiple working channels defined therein.

### Description of Related Art

A wide variety of catheters, as well as surgical instruments designed to be used with such devices, have been developed. Of these known devices, each has certain advantages and disadvantages. However, there is an ongoing need to provide alternative catheters. For example, in some instances, some known catheters may only include one working channel configured to receive only one surgical instrument therein at a time. In surgical procedures requiring the use of multiple instruments, a catheter having only one working channel may require the removal and replacement of the various instruments throughout the procedure which may not only time consuming, but also may increase the likelihood of the catheter shifting out of position during removal and/or replacement of the various surgical instruments. In addition, a single circular working channel may prevent the use of two or more instruments at the same time. Thus, there exists a need to provide catheters having multiple working channels to provide access and/or use of multiple instruments simultaneously at a target tissue.

### SUMMARY

The present disclosure describes catheters including multiple working channels. In some embodiments, the multiple working channels include two asymmetrical working channels.

The present disclosure also describes a catheter including a shaft portion defining a non-circular transverse cross-section. The shaft portion includes an exterior sidewall and an interior sidewall defining two or more asymmetrical working channels therein.

In some embodiments, the non-circular transverse cross-section of the catheter defines an elliptical transverse cross-section. The elliptical transverse cross-section of the shaft includes a major axis and a minor axis which cross at a central portion of the elliptical cross-section.

In some embodiments, the multiple working channels include a first and second working channel. In some embodiments, the first working channel generally defines a circular transverse cross-section and the second working channel generally defines a non-circular transverse cross-section. In some embodiments, the first working channel defines a generally reniform transverse cross-section and the second working channel defines a non-reniform transverse cross-section.

In some embodiments, the catheters described herein include a handle portion on a proximal end and a shaft portion on a distal end thereof. The shaft portion includes an exterior wall and an interior sidewall. The exterior sidewall defines an outer elliptical transverse cross-section and the interior sidewall defines a first and second working channel within the exterior wall. The first working channel defines a generally circular transverse cross-section and the second working channel defines a generally reniform transverse cross-section.

Use of the catheters described herein electromagnetic navigation systems for navigating through a luminal network of a patient, and particularly of a patient's lung, is also provided herein.

### BRIEF DESCRIPTION OF THE DRAWINGS

Various embodiments are described herein by way of example in conjunction with the following:

FIG. 1A is a schematic side view of a multi-lumen catheter as described in at least one embodiment herein:

FIG. 1B is a transverse cross-sectional view of a multi-lumen catheter as described in at least one embodiment herein; and

FIG. 2 is a schematic perspective view of a navigation system for visualizing a lung of a patient as described in at least one embodiment herein.

### DETAILED DESCRIPTION

Detailed embodiments of the present disclosure are disclosed herein; however, the disclosed embodiments are merely examples of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Aspects of the present disclosure are described in detail with reference to the drawing figures wherein like reference numerals identify similar or identical elements. As used herein, the term “distal” refers to the portion that is being described which is further from a user, while the term “proximal” refers to the portion that is being described which is closer to a user.

The present disclosure is directed in part to a catheter including a shaft portion defining a non-circular transverse cross-section and including an exterior and interior wall combining to define two or more asymmetrical working channels defined therein. Each working channel being configured to receive and/or pass therethrough a surgical instrument. The shaft portion and/or the working channels being configured for navigation within a luminal structure of a patient.

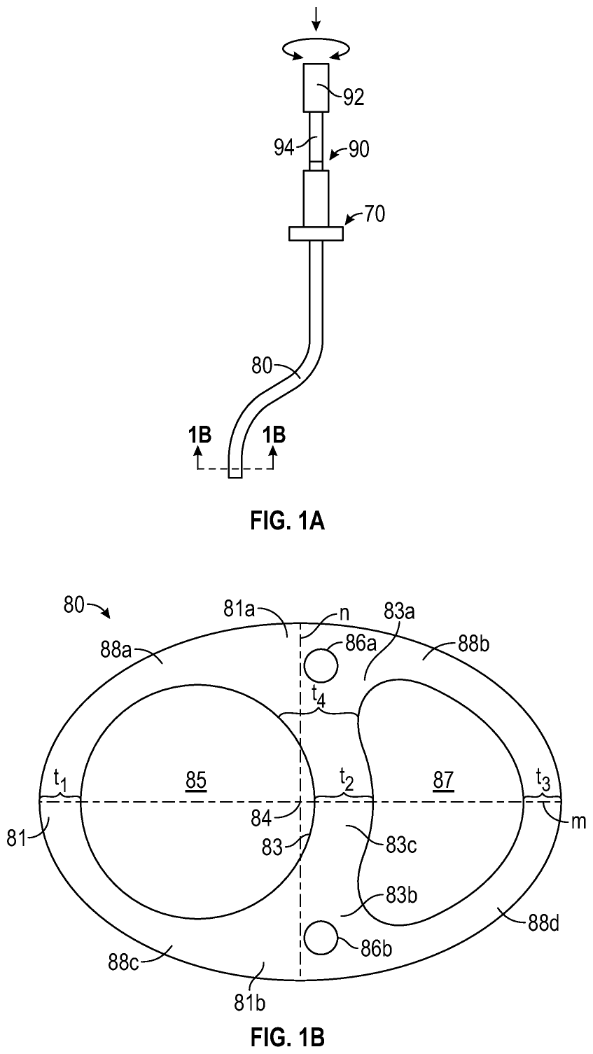

FIG. 1A depicts a catheter 70 as described herein including a handle portion 90 on a proximal end thereof and a shaft portion 80 extending in a distal direction from the handle portion 90. Any suitable handle portion configuration may be used with the shaft portion described herein. For example, the handle portion may be manually operated or power operated, both of which are generally known in the art.

In some embodiments, as shown in FIG. 1A, the handle portion 90 may be manually operated and include at least one grip 92 and a telescopic connector 94, which are operably connected to the shaft portion 80. By rotating the grip 92 and translating the telescopic shaft 94, the user is able to steer at least the distal end portion, if not a majority or all, of the shaft portion 80 to a target tissue using one or both hands. These movements of the handle 90 enable the user to navigate at least the distal end portion of the shaft portion 80 through the tortuous path of a luminal network such as the patient airways, and direct advancement of at least the distal end portion of the shaft portion 80 at each bifurcation. An example of such a handle 90 is currently manufactured and sold under the name EDGE™ by Covidien LP. The handle 90 may be ergonomically shaped to facilitate grasping and/or rotation of shaft portion 80. In one embodiment, a plurality of rib portions (not explicitly shown) are provided along a length of the handle 90 to facilitate grasping and rotation of the shaft portion 80.

FIG. 1B depicts the shaft portion 80 defining a non-circular cross-section transverse to a longitudinal axis of the catheter 70. The non-circular transverse cross-section of the shaft portion 80 may define any non-circular shape including but not limited to elliptical, hexagonal, pentagonal, heptagonal, octagonal, trapezoidal, etc. It is envisioned that the non-circular transverse cross-section may enhance the surgical instruments ability to travel through a selected luminal network which typically may be circular in cross-section.

In some embodiments, as further depicted in FIG. 1B, the non-circular cross-section of the shaft portion 80 defines an elliptical transverse cross-section. An elliptical cross-section defines a major axis m and a minor axis n which cross at generally a center 84 of the elliptical transverse cross-section. The major axis m and the minor axis n define the elliptical cross-section into a first and second upper quadrant 88a, 88b and a first and second lower quadrant 88c, 88d of the elliptical transverse cross-section. As particularly shown in FIG. 1B, in some embodiments, the major axis m may be a lateral major axis and the minor axis n may be a vertical minor axis. However, it is envisioned that in some embodiments the major axis m may be a major vertical axis and the minor axis n may be a minor lateral axis.

FIG. 1B further depicts shaft portion 80 including an exterior sidewall 81 and interior wall 83. The exterior sidewall 81 defines an outer perimeter of the shaft portion 80. The interior sidewall 83 includes a first end portion 83a, a second end portion 83b opposite the first end portion 83a and body portion 83c positioned therebetween. The first end portion 83a is connected to a first portion 81a of the exterior sidewall 81 and the second end portion 83b is connected to a second portion 81b of the exterior sidewall 81. The body portion 83c extends therebetween defining, along with the exterior sidewall 81, the two or more asymmetrical working channels 85, 87. In some embodiments, the body portion 83c of the inner sidewall 83 is curved and offset from the minor axis n.

By asymmetrical, the two or more working channels 85, 87 define any combination of at least two different transverse cross-sectional shapes. For example, one working channel 85 may define a generally circular cross-section and another working channel 87 may define a non-circular cross-section. In some embodiments, the asymmetrical working channels 85, 87 may further be arranged off-center, i.e., not equally distanced from a center of the non-circular transverse cross-section.

As further shown in FIG. 1B, in some embodiments, the two asymmetrical working channels 85, 87 may include a first and second working channels 85, 87. At least a portion of the first working channel 85 may extend across the center 84 of the non-circular, e.g., elliptical, transverse cross-section. In some embodiments, at least a portion of the first working channel 85 may extend across at least a portion of each of the first and second upper quadrants 88a, 88b and the first and second lower quadrants 88c, 88d of the non-circular, e.g., elliptical, transverse cross-section. In some embodiments, at least a portion of the second working channel 87 may extend across only the second upper and lower quadrants 88b, 88d of the non-circular, e.g., elliptical, transverse cross-section. The two or more working channels 85, 87 may further each be centered on the major axis m and not centered on the minor axis n.

FIG. 1B further illustrates that the first working channel 85 may define a circular transverse cross-section and/or the second working channel 87 may define a non-circular, e.g., reniform, transverse cross-section. It is envisioned that the non-circular transverse cross-section of the second working channel may be utilized with surgical instruments defining the same non-circular cross-section. For example, both the second working channel and a surgical instrument (e.g., a locatable guide, ultrasound probe, or camera) may define a non-circular, e.g., reniform, cross-section. In such an example, the mating of the two non-circular, e.g., reniform, cross-sections of the channel and the instrument can lock the instrument into a predetermined configuration relative to the other working channels because the instrument is unable to rotate within the channel, as is possible when both the instrument and the channel define a circular cross-section.

In some embodiments, a first thickness t₁ of the exterior sidewall 81 and a second thickness t₂ of the interior sidewall 83 may be generally equal, i.e., t₁=t₂, along the major axis m of the non-circular, e.g., elliptical, transverse cross-section and/or may be generally unequal along the minor axis n of the non-circular, e.g., elliptical, transverse cross-section. In some embodiments, the first thickness t₁ of the exterior sidewall 81 is generally uniform throughout, i.e., t₁=t₃, and/or a thickness of the interior sidewall is generally non-uniform throughout, i.e., t₂ is not equal to t₄.

As further shown in FIG. 1B, in some embodiments, the shaft portion 80 may further include one or more lumens 86a, 86b defined therethrough. The lumens being smaller in dimension than the two or more working channels 85, 87. The working channels 85, 87 being configured to accommodate a variety of surgical instruments and guides of varying dimensions. The lumens 86a, 86b being unable to accommodate the surgical instruments described herein. However, the lumens 86a, 86b may be individually configured to provide suction therethrough, the ability to inject a substance therethrough, a light source therethrough, or even a guidewire therethrough for aiding in steering of the shaft portion 80. The lumens 86a, 86b are shown defining a generally circular cross-section. However, other non-circular cross-section configurations may also be used.

The catheters described herein may include a handle portion on a proximal end and a shaft portion on a distal end thereof, the shaft portion including an exterior wall and an interior sidewall, the exterior sidewall defining a non-circular, e.g., elliptical, transverse cross-section and the interior sidewall defining a first and second working channel within the exterior wall, the first working channel defining a generally circular transverse cross-section and the second working channel defining a generally reniform transverse cross-section.

The catheters, and particularly the shaft portions of the catheters, described herein may be formed from any suitable biocompatible material, including but not limited to, rubbers and plastics acceptable for surgical and medical use. It is contemplated that a suitable material (not specifically shown) may include a braided support structure formed from metals, such as stainless steel, or alternatively one or more non-conductive fibrous materials such as Kevlar or other aramid fibers to provide additional resilience and to maintain one or more of the working channels in a generally open configuration to ease the passage of the multiple surgical instruments therethrough.

The catheters, and particularly the shaft portions of the catheters, described herein may be formed using any suitable method, including but not limited to, molding, extrusion, pressing, casting, and the like.

In addition, although not shown, in some embodiments, the handle portion of the catheters described herein may include one large channel configured to accommodate the multiple surgical instruments positioned with the working channels or alternatively the handle may include multiple channels configured to accommodate any combination of the multiple working channels therethrough.

The catheters as described herein are configured to be used with systems for visualizing a luminal network of a patient, and/or particularly a lung of a patient. The systems and/or catheters as described herein may use ultrasound (US) imaging technologies which provide a sufficient resolution to identify and locate a target for diagnostic, navigation, and treatment purposes. US imaging, particularly in conjunction with non-invasive imaging, can provide a greater resolution and enable luminal network mapping and target identification. Further, additional clarity is provided with respect to tissue adjacent the endoscope, catheter, or identified targets which can result in different treatment options being considered to avoid adversely affecting the adjacent tissue.

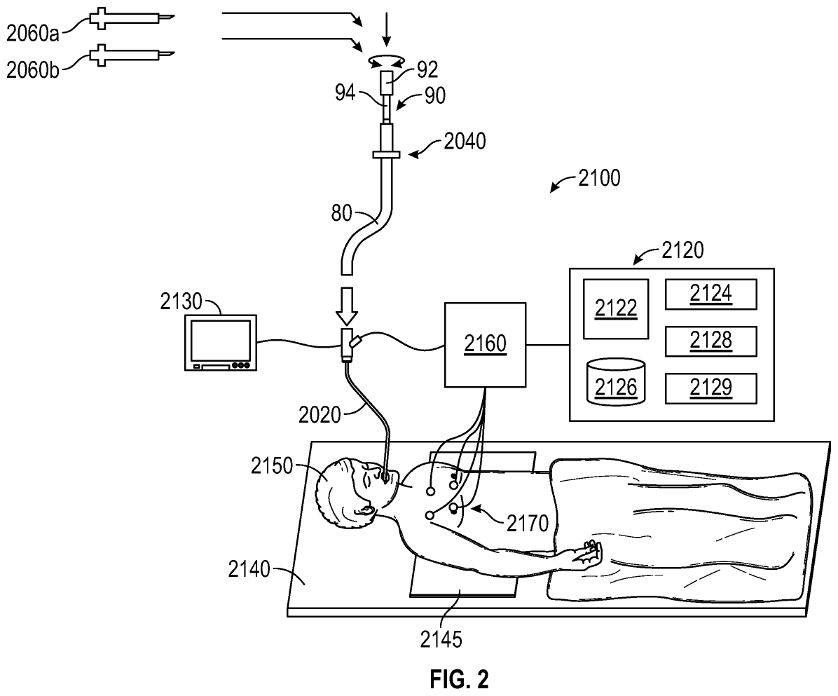

FIG. 2 illustrates an electromagnetic navigation (EMN) system 2100, which is configured to augment CT, MRI, or fluoroscopic images, with US image data assisting in navigation through a luminal network of a patient's lung to a target. One such EMN system may be the ELECTROMAGNETIC NAVIGATION BRONCHOSCOPY® system currently sold by Covidien LP. The system 2100 includes an endoscope assembly 2010 including an endoscope 2020, a catheter 2040 as described herein including a shaft 80 with two or more asymmetrical working channels, multiple surgical instruments 2060a, 2060b, a computing device 2120, a monitoring device 2130, an EM board 2140, a tracking device 2160, and reference sensors 2170. The endoscope 2020 is specifically a bronchoscope which is operatively coupled to the computing device 2120 and the monitoring device 2130 via wired connection (as shown in FIG. 2) or wireless connection (not shown).

The bronchoscope 2020 is inserted into the mouth of the patient 2150 and captures images of the luminal network of the lung. In the EMN system 2100, inserted into the bronchoscope 2020 is a catheter 2040 as described herein for achieving access to the periphery of the luminal network of the patient 2150. The catheter 2040 includes a shaft portion 80 as described herein including two or more asymmetric working channels into which multiple surgical instrument 2060a, 2060b may be inserted. A first surgical instrument 2060a, such as a locatable guide including an electromagnetic (EM) sensor at the distal tip thereof, may be inserted into one of the two or more working channels to help navigate through the luminal network of the lung as described in greater detail below. Because the catheter includes at least a second working channel, a second surgical instrument 2060b, such a cytology brush, biopsy needle, or ablation catheter, may be inserted into the a second of the two or more working channels and navigated through the luminal network of the lungs with the first instrument 2060a. Therefore, upon arrival of a desired location in the lung, the locatable guide may not need to be removed from the working channel to and replaced with the second surgical instrument 2060b. However, it is envisioned that the locatable guide may be removed and replaced with a third surgical instrument designed to work with the second surgical instrument to perform multiple steps of the procedure simultaneously.

The computing device 2120, such as, a laptop, desktop, tablet, or other similar computing device, includes a display 2122, one or more processors 2124, memory 2126, a network card 2128, and an input device 2129. The system 2100 may also include multiple computing devices, wherein the multiple computing devices 2120 are employed for planning, treatment, visualization, or helping clinicians in a manner suitable for medical operations. The display 2122 may be touch-sensitive and/or voice-activated, enabling the display 2122 to serve as both an input and output device. The display 2122 may display a two dimensional (2D) images or three dimensional (3D) model of a luminal network, such as found in the lung, to locate and identify a portion of the network that displays symptoms of disease, such as lung disease. The generation of such images and models is described in greater detail below. The display 2122 may further display options to select, add, and remove a target to be treated and settable items for the visualization of the network or lung. In an aspect, the display 2122 may also display the location of the catheter 2040 in the luminal network of the lung based on the 2D images or 3D model of the lung. For ease of description not intended to be limiting on the scope of this disclosure, a 3D model is described in detail below but one of skill in the art will recognize that similar features and tasks can be accomplished with 2D models and images.

The one or more processors 2124 execute computer-executable instructions. The processors 2124 may perform image-processing functions so that the 3D model of the lung can be displayed on the display 2122. In embodiments, the computing device 2120 may further include a separate graphic accelerator (not shown) that performs only the image-processing functions so that the one or more processors 2124 may be available for other programs.

The memory 2126 stores data and programs. For example, data may be image data for the 3D model or any other related data such as patients' medical records, prescriptions and/or history of the patient's diseases. One type of programs stored in the memory 2126 is a 3D model and pathway planning software module (planning software). An example of the 3D model generation and pathway planning software may be the ILOGIC® planning suite currently sold by Covidien LP. When image data of a patient, which is typically in digital imaging and communications in medicine (DICOM) format, from for example a CT image data set (or image data set by other imaging modality) is imported into the planning software, a 3D model of the bronchial tree is generated. In an aspect, imaging may be done by CT imaging, magnetic resonance imaging (MRI), functional MRI, X-ray, and/or any other imaging modalities. To generate the 3D model, the planning software employs segmentation, surface rendering, and/or volume rendering. The planning software then allows for the 3D model to be sliced or manipulated into a number of different views including axial, coronal, and sagittal views that are commonly used to review the original image data. These different views allow the user to review all of the image data and identify potential targets in the images.

Once a target is identified, the software enters into a pathway planning module. The pathway planning module develops a pathway plan to achieve access to the targets and the pathway plan pin-points the location and identifies the coordinates of the target such that they can be arrived at using the EMN system 2100 in combination with any of the catheters described herein, and particularly the catheter 2040 and a first and second surgical instrument 2060a, 2060b. The pathway planning module guides a clinician through a series of steps to develop a pathway plan for export and later use in during navigation to the target in the patient 2150. The term, clinician, may include doctor, surgeon, nurse, medical assistant, or any user of the pathway planning module involved in planning, performing, monitoring and/or supervising a medical procedure.

The memory 2126 may store navigation and procedure software which interfaces with the EMN system 2100 to provide guidance to the clinician and provide a representation of the planned pathway on the 3D model and 2D images derived from the 3D model. An example of such navigation software may be the ILOGIC® navigation and procedure suite sold by Covidien LP. In practice, the location of the patient 2150 in the EM field generated by the EM field generating device 2145 must be registered to the 3D model and the 2D images derived from the model. Such registration may be manual or automatic.

As further shown in FIG. 2, the EM board 2140 is configured to provide a flat surface for the patient to lie down and includes an EM field generating device 2145. When the patient 2150 lies down on the EM board 2140, the EM field generating device 2145 generates an EM field sufficient to surround a portion of the patient 2150. An EM sensor on a distal tip of the LG 2060a may be used to determine the location of the LG 2060a in the EM field generated by the EM field generating device 2145.

In some embodiments, the EM board 2140 may be configured to be operatively coupled with the reference sensors 2170 which are located on the chest of the patient 2170. The reference sensors 2170 move up and down following the chest while the patient 2150 is inhaling and move down following the chest while the patient 2150 is exhaling. The movement of the reference sensors 2170 in the EM field is captured by the reference sensors 2170 and transmitted to the tracking device 2160 so that the breathing pattern of the patient 2150 may be recognized. The tracking device 2160 also receives outputs of the EM sensor on the LG 2060a, combines both outputs, and compensates the breathing pattern for the location of the LG 2060a. In this way, the location identified may be compensated for so that the compensated location of the LG 2060a is synchronized with the 3D model of the lung. Once the patient 2150 is registered to the 3D model, the position of the catheter 2040 described herein and particularly the LG 2060a and the second surgical instrument 2060b, can be tracked within the EM field generated by the EM field generator 2145, and the position of the LG 2060a can be depicted in the 3D model or 2D images of the navigation and procedure software.

When the endoscope 2020 or catheter 2040, and the LG 2060a, reaches a target tissue by following the pathway plan, the LG 2060a including the EM sensor confirms its location at the target and a clinician may confirm the location at the target. Since the second instrument 2060b was included within the catheter 2040, the general location of the second instrument 2060b may also be confirmed. The LG 2060a may either remain in the catheter 2040 or be removed from the catheter 2040 after arriving at the target tissue. In some embodiments, the LG 2060a may be withdrawn and replaced with a third surgical instrument (not shown) configured to work together with the second instrument 2060b to treat the tissue or retrieve a sample of the target for confirmation of the disease.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting but merely as exemplifications of particular embodiments. Those skilled in the art will envision other modifications within the scope and spirit of the claims appended hereto.

1. A catheter comprising a shaft portion defining a non-circular transverse cross-section, the shaft portion including an exterior sidewall and an interior sidewall defining two or more asymmetrical working channels therein.

2. The catheter of claim 1, wherein the non-circular transverse cross-section defines an elliptical transverse cross-section.

3. The catheter of claim 2, wherein the elliptical cross-section of the shaft portion includes a major axis and a minor axis which cross at a center of the elliptical transverse cross-section, the major axis and the minor axis defining a first and second upper quadrant and a first and second lower quadrant of the elliptical transverse cross-section.

4. The catheter of claim 3, wherein a thickness of the exterior sidewall and a thickness of the interior sidewall is equal along the major axis.

5. The catheter of claim 3, wherein a thickness of the exterior sidewall is generally uniform throughout and a thickness of the interior sidewall is generally non-uniform.

6. The catheter of claim 3, wherein the interior sidewall includes a first end portion, a second end portion opposite the first end portion and body portion positioned therebetween, the first end portion connected to a first portion of the exterior sidewall and the second end portion connected to a second portion of the exterior sidewall.

7. The catheter of claim 6, wherein the body portion of the inner sidewall is curved and offset from the minor axis.

8. The catheter of claim 3, wherein the two or more working channels include a first working channel including a circular transverse cross-section and a second working channel including a non-circular transverse cross-section.

9. The catheter of claim 3, wherein the two or more working channels include a first working channel including a circular transverse cross-section and a second working channel including a reniform transverse cross-section.

10. The catheter of claim 8, wherein the first and second working channels are each centered on the lateral major axis and not centered on the vertical minor axis.

11. The catheter of claim 8, wherein at least a portion of the first working channel is positioned on the central portion of the elliptical transverse cross-section.

12. The catheter of claim 8, wherein at least a portion of the first working channel is positioned in each of the first and second upper quadrants and the first and second lower quadrants of the elliptical transverse cross-section.

13. The catheter of claim 8, further comprising at least a first lumen and a second lumen extending therethrough.

14. The catheter of claim 13, wherein the second working channel, the first lumen and the second lumen are positioned only in the second upper and lower quadrants of the elliptical transverse cross-section.

15. A catheter comprising a handle portion on a proximal end and a shaft portion on a distal end thereof, the shaft portion including an exterior wall and an interior sidewall, the exterior sidewall defining an elliptical transverse cross-section and the interior sidewall defining a first and second working channel within the exterior wall, the first working channel defining a generally circular transverse cross-section and the second working channel defining a generally reniform transverse cross-section.

16. An electromagnetic navigation system for navigating through a luminal network of a patient's lung, the system comprising:
a computing device,
a monitoring device,
an electromagnetic board,
a tracking device, and
a catheter including a shaft portion defining a non-circular transverse cross-section, the shaft portion including an exterior sidewall and an interior sidewall defining two or more asymmetrical working channels therein, and optionally at least one of a bronchoscope or surgical instrument.
